(19) United States
(12) Patent Application Publication
(10) Pub. No.: US 2025/0161611 A1
(43) Pub. Date: May 22, 2025

(54) PATIENT INTERFACE INCLUDING TRIBOELECTRIC SENSOR

(71) Applicant: KONINKLIJKE PHILIPS N.V., EINDHOVEN (NL)

(72) Inventors: DAAN ANTON VAN DEN ENDE, BREDA (NL); JOYCE VAN ZANTEN, WAALRE (NL); CORNELIS PETRUS HENDRIKS, EINDHOVEN (NL); JOACHIM JOHANNES KAHLERT, AACHEN (DE); JONATHAN SAYER GRASHOW, CHESWICK, PA (US)

(21) Appl. No.: 18/949,253

(22) Filed: Nov. 15, 2024

Related U.S. Application Data
(60) Provisional application No. 63/600,074, filed on Nov. 17, 2023.

(51) Int. Cl. A61M 16/06; A61M 16/00

(52) U.S. Cl. CPC A61M 16/0605; A61M 16/0003; A61M 16/024; A61M 2202/0007; A61M 2202/02; A61M 2205/3331; A61M 2205/3334; A61M 2210/0618

(57) ABSTRACT

A patient interface for use in providing a flow of a pressurized treatment gas to an airway of a patient includes a body for receiving the flow of the pressurized treatment gas, a sealing member for engaging with the face of the patient about the airway of the patient, and at least one triboelectric sensor configured to provide a signal indicative of information about one or more of the body and/or the sealing member.

### CROSS-REFERENCE TO RELATED APPLICATIONS

This patent application claims the priority benefit under 35 U.S.C. § 119(e) of U.S. Provisional Application No. 63/600,074, filed on Nov. 17, 2023, the contents of which are herein incorporated by reference.

### BACKGROUND OF THE INVENTION

### 1. Field of the Invention

The disclosed concept relates generally to patient interfaces for use in providing a flow of a pressurized treatment gas to an airway of a patient, and more particularly to patient interface devices including triboelectric self-powered sensors for use in determining information about the patient interface. The disclosed concept further relates to systems and methods for determining information regarding a patient interface from triboelectric sensors included on the patient interface.

### 2. Description of the Related Art

Today, the first line of therapy for patients diagnosed with obstructive sleep apnea syndrome (OSAS) after a sleep test is a pressure assisted ventilation support, most often by continuous positive airway pressure (CPAP) therapy. In moderate and severe patients with an AHI>15, the therapy is reimbursed by insurance. In mild OSA patients with daytime symptoms, or with chronic and persistent cardiac comorbidities, the PAP therapy is reimbursed for an AHI>5. Reimbursement covers the PAP device as well as periodic resupply of consumable items, such as tubing, headgear, masks and cushions. Depending on geography, different time periods for replacement of these consumable items are in effect, mostly ranging from 1 month to 6 months.

The proper setup of the PAP device, including for instance pressure settings and fitting of the mask/patient interface, is done by a qualified sleep clinician, most often in an overnight setting at a sleep lab, although home titration is a possible alternative for certain patients. Once the correct machine settings and appropriate consumable items are established, the equipment is supplied by a durable medical equipment (DME) supplier and the patient commences therapy. In the event of difficulties with any aspects of the therapy, a consultation, optionally followed by re-titration may be performed, which may result in a change in mask type. For instance, a patient may switch from a nasal mask to an oronasal or full-face mask. This change has to be communicated with the DME, who then provides the new mask to the patient.

Incidentally, patients may also buy a mask/patient interface out-of-pocket. For instance, if the mask is lost or broken in between reimbursement periods, or if the patient believes that they need a different mask than was advised by the clinician. As a result, the patient may end up using a different mask than was specified by the clinician for their therapy, potentially causing a mismatch in the mask that is used compared to the specified mask, which potentially has great influence on the therapy provided to the patient because the PAP machine settings are no longer matching the mask.

The identification of the actual mask that is used during therapy at any given time is a need that is expressed by clinicians and DMEs alike. Also, when patients use different mask interchangeably (for instance patients switching between nasal and oronasal masks) the optimal pressure and flow settings will differ and therefore the problem arises that the PAP machine must be manually reconfigured when switching masks.

Several approaches have attempted to address the mask identification issue with proposed solutions for automatic identification considering the use of electronic tags (U.S. Pat. Nos. 8,695,602B2, 10,124,141B2, 7,191,780B2, 8,766,790B2) and identification through electrical means by various add-on elements (U.S. Pat. No. 10,279,134B2). Other means of identification such as bar/QR-code (DE102015003953A1) or serial number scanning (US2016321420A1) have also been proposed. Many of these inventions relate to the automatic adjustment of settings, depending on the type of mask that is used. Furthermore, from market research studies, many clinicians also indicate the need to know which mask is being used by the patient in order to be able to diagnose problems more accurately (especially in a remote/telehealth setting).

While such proposed solutions address the need for identification of the patient interface elements in some form, the use of electronic tags and/or an add-on element for electrical identification require providing power to the mask, in wired or wireless form, which increases complexity in the design and adds cost to the device. If the tag is an active tag, it may be battery powered, which introduces the risk of a depleted battery causing malfunctioning of the device and misidentification. Scanning of QR codes and/or serial numbers is often not an automatic process but requires user input, which again is an error-prone process as it ultimately relies on the user to take action whenever a mask is changed.

Another problem that exists is identifying when a particular mask is wearing out and thus should be replaced. Masks are available in many different styles and sizes and each mask (element) may have a different wear out profile, for instance a nasal mask cushion may wear out faster than that of a full face mask or a mask with foam elements may need to ultimately be replaced within a period of time, before the foam starts to degrade and lose its functionality. Additionally, the use condition and/or number of washing cycles may greatly influence mask degradation, rendering an average recommended use time inaccurate (at least for some patients). Hence, a method of automatic mask type logging and wear-out monitoring would be very beneficial to give a true and accurate mask wear-out alert to the patient and verifying if the patient changes their mask when instructed to.

Several approaches have been made to address such issue(s). For instance, US2016321420 A1, discloses the use of sensors which can accurately determine the use time from face contact measurement and also by sensing degradation of the headgear straps. Another example is US2009199857 A1, which discloses a reminder system for a patient to service or replace their mask using data from a sensor which measures predetermined properties of the cushion (for instance, the presence of lipids or other bodily fluids may change the conductivity of the cushion) and generates a signal when a threshold is reached. In another example, the ability to change color may wash out after a number of washes prompting the patient to obtain a new mask.

While such arrangements generally provide at least some solutions for the aforementioned problems, there is still room, and a further need, for better solutions.

### SUMMARY OF THE INVENTION

Embodiments of the disclosed concept address the aforementioned problems and shortcomings of known solutions by utilizing arrangements including triboelectric sensors. As one aspect of the disclosed concept, a patient interface for use in providing a flow of a pressurized treatment gas to an airway of a patient is provided. The patient interface comprises: a body structured to receive the flow of the pressurized treatment gas; a sealing member structured to engage with the face of the patient about the airway of the patient; and at least one triboelectric sensor structured to provide a signal indicative of information about one or more of the body and/or the sealing member.

The at least one triboelectric sensor may be structured to provide the signal indicative of information about one or more of the body and/or the sealing member when a portion of the patient interface moves with respect to another portion of the patient interface or with respect to the face of the patient. The signal may be indicative of a type of patient interface or a portion thereof. The signal may be indicative of a size of the patient interface or a portion thereof. The signal may be indicative of a condition of a portion of the patient interface. The patient interface may further comprise an antenna in communication with the at least one triboelectric sensor. The at least one triboelectric sensor may comprise a plurality of triboelectric sensors. At least two triboelectric sensors of the plurality of triboelectric sensors may be structured to provide a signal indicative of the same information. At least two triboelectric sensors of the plurality of triboelectric sensors may be structured to provide a signal indicative of different information.

As another aspect of the disclosed concept, a system for providing a flow of a pressurized treatment gas to an airway of a patient is provided. The system comprises: a pressure generating device structured to produce a flow of a pressurized treatment gas; a patient interface coupled to, and structured to receive the flow of pressurized treatment gas from, the pressure generating device via a conduit, the patient interface comprising: a body structured to receive the flow of the pressurized treatment gas from the conduit; a sealing member structured to engage with the face of the patient about the airway of the patient; and at least one triboelectric sensor structured to provide a signal indicative of information about one or more of the body and/or the sealing member; and a controller in communication with the at least one triboelectric sensor, the controller structured to receive the signal indicative of information about one or more of the body and/or the sealing member and determine therefrom the information about the one or more of the body and/or the sealing member.

The at least one triboelectric sensor may be structured to provide the signal indicative of information about one or more of the body and/or the sealing member when a portion of the patient interface moves with respect to another portion of the patient interface or with respect to the face of the patient. The signal may be indicative of: a type of patient interface or a portion thereof, a size of the patient interface or a portion thereof, or a condition of a portion of the patient interface. The system may further comprise an antenna in communication with the at least one triboelectric sensor. The at least one triboelectric sensor may comprise a plurality of triboelectric sensors.

As yet a further aspect of the disclosed concept, a method of determining information about a patient interface used in providing a flow of pressurized treatment gas to the airway of a patient is provided. The method comprises: receiving a signal from a triboelectric sensor provided on the patient interface; comparing the signal to a database of known signals; determining information about the patient interface from the comparison; and providing an output based on the information.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

A full understanding of the invention can be gained from the following

description of the preferred embodiments when read in conjunction with the accompanying drawings in which:

FIGS. 1A-1D are schematic representations of the four main triboelectric generation modes;

FIG. 2 is partially schematic view of a system in accordance with an example embodiment of the disclosed concept;

FIG. 3 is a flowchart showing steps of a method (along with related events) employing one or more triboelectric sensors positioned on a patient interface in accordance with an example embodiment of the disclosed concept;

FIG. 4A is an example embodiment of a triboelectric sensor arrangement in accordance with an example embodiment of the disclosed concept;

FIG. 4B is a schematic view of the triboelectric sensor components of the arrangement of FIG. 4A;

FIG. 4C shows schematic representations of the triboelectric sensor components of FIG. 4B positioned in different relative positions with respect to each other in accordance with example embodiments of the disclosed concept; and

FIG. 5 is a schematic representation of an example circuit for wireless transmission of a triboelectric signal in accordance with an example embodiment of the disclosed concept.

### DETAILED DESCRIPTION OF THE INVENTION

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

As used herein, the statement that two or more parts or components “engage” one another shall means that the parts exert a force against one another either directly (i.e., “directly engage”) or through one or more intermediate parts or components. As used herein, the word “unitary” means a component is created as a single piece or unit. That is, a component that includes pieces that are created separately and then coupled together as a unit is not a “unitary” component or body. As used herein, the term “number” shall mean one or an integer greater than one (i.e., a plurality).

As used herein, “mask” and “patient interface” are used interchangeably and, as such, neither of such terms are intended to be broader or limiting than the other.

As discussed in greater detail below, embodiments of the disclosed concept utilize triboelectric sensor arrangements to provide information regarding a particular patient interface. Triboelectricity to some extent is present between all material combinations. When two different materials in contact with each other are separated they become electrically charged (static electricity). Reciprocal movement can enhance the (transient) contact between the materials and cause a higher charge buildup in the two separated materials. This charge can be collected by electrodes near the surface of the materials and used as an electric signal for the displacement of the materials. Types of materials can be ranked in a triboelectric series where some materials have affinity for positive charging while others become negatively charged upon separation. The amount of charge that is developed when the materials are separated is increased when the material combination consists of two materials on opposite ends of the spectrum.

Triboelectric sensors can be made using extremely simple configurations

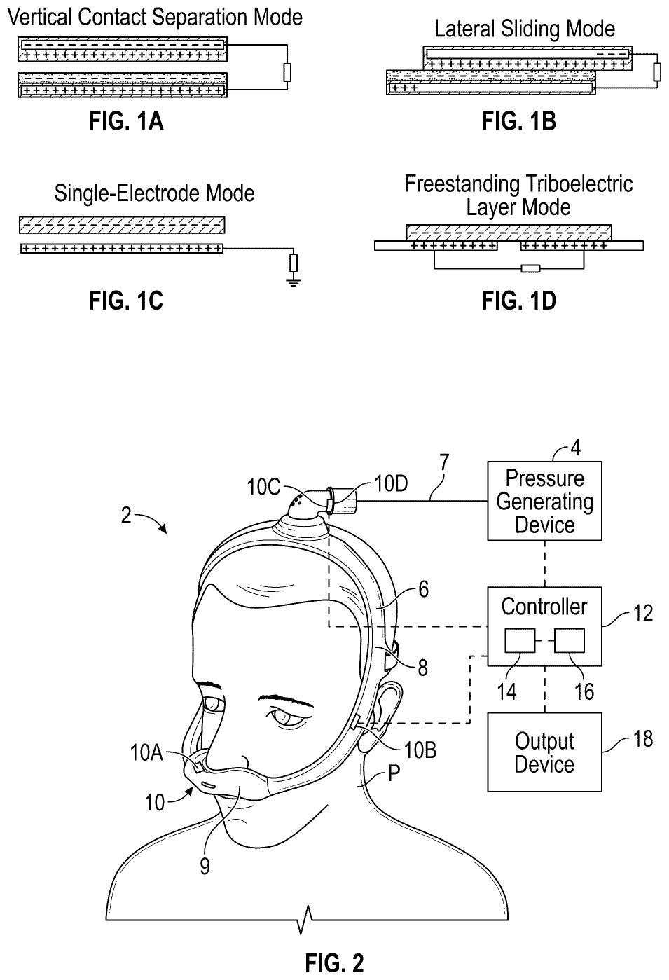

and low-cost materials and are easily compatible with current products and manufacturing methods. Human skin for instance is a highly triboelectric material which is known to be highly affinitive with positive charging while for instance materials such as polyimide, PTFE (Teflon) and silicone rubber predominantly produce negative triboelectric charges. In addition to being simple and low-cost, triboelectric sensors also have the added benefit of being self-powered (i.e., no external power source is needed) and as such the sensors can also be used as micro power generators. Triboelectric sensors have gained popularity in recent years and many examples of triboelectric micro generators or self-powered sensors are known, including systems making use of sheets, films and foams as triboelectric materials and charge transfer mechanisms including contact separation mode and sliding modes or a combination of both. Referring to FIG. 1, there are four main triboelectric generation modes: (A) vertical contact separation mode, (B) lateral sliding mode, (C) single electrode mode and (D) freestanding triboelectric layer mode.

Triboelectric signals are susceptible to variation due to the nature of the signal generation. The exact mechanism of electricity generation is still not fully understood, although it is clear that the nature of the contact, the state of the surface of the material(s) and the environmental conditions all influence the output. Also, triboelectric signals are typically high impedance signals which may be susceptible to noise on the output which must be accommodated for in any design using triboelectricity as a sensor mechanism.

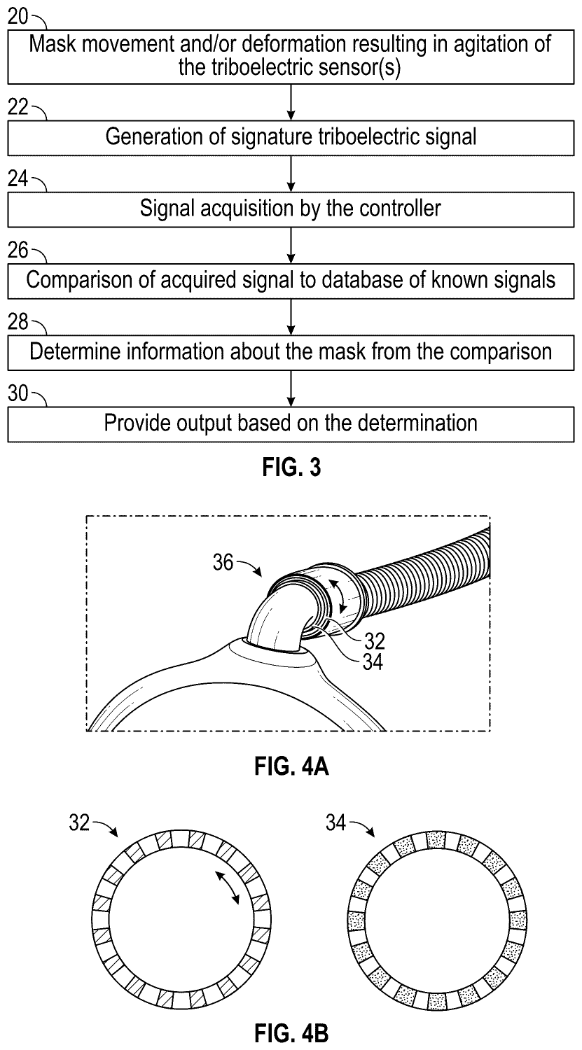

Embodiments of the disclosed concept utilize triboelectric sensor arrangements positioned on a mask/patient interface used in delivering a flow of pressurized treatment gas to the airway of a patient. Depending on the particular application, the triboelectric sensors are positioned so as to generate a signal when the patient interface is moved and/or deformed. For example, the triboelectric sensor(s) can be positioned so as to produce a signal when movement between the patient interface and patient occurs (e.g., when the patient interface is put on the face by the patient before commencing therapy, when movement occurs during treatment) and/or when movement/deformation/intermittent contact between parts of the patient interface occurs. A system 2 in accordance with one example embodiment of the disclosed concept is shown in FIG. 2, while a flowchart showing steps of a method employing one or more triboelectric sensors positioned on a patient interface mask along with related events in accordance with an example embodiment of the disclosed concept is shown in FIG. 3.

Referring to FIG. 2, system 2 includes a pressure generating device 4 (shown schematically) coupled to a patient interface 6 via a conduit 7 (also shown schematically). Pressure generating device 4 is structured to produce a flow of a pressurized treatment gas which is communicated to patient interface 6 via conduit 7 for delivery to an airway of a patient P. Patient interface 6 includes a body 8 that is structured to receive the flow of the pressurized treatment gas from conduit 7 and a sealing member 9 structured to engage with the face of the patient about the airway of the patient. While patient interface 6 shown in the example embodiment of FIG. 2 is similar to the Philips Respironics DreamWear Nasal CPAP Mask, it is to be appreciated that such patient interface is provided for exemplary purposes only and that other patient interface arrangements may be employed without varying form the scope of the disclosed concept.

Continuing to refer to FIG. 2, patient interface 6 further includes a number of triboelectric sensors 10 (four are shown schematically labeled 10A-10D) in communication (e.g., via any suitable wired or wireless arrangement, discussed further below) with a controller 12 provided as a part of system 2. Controller 12 includes a processing portion 14 which may be, for example, a microprocessor, a microcontroller or some other suitable processing device or devices, and a memory portion 16 that may be internal to processing portion 14 or operatively coupled to processing portion 14 and that provides a storage medium for data and software executable by the processing portion 14. An output device 18 (e.g., a display or any other suitable arrangement may be provided as a part of system 2 for providing output from controller 12. Additionally or alternatively, output from controller 12 may be provided to other arrangements as discussed further below. Controller 12 may be provided as a component separate from pressure generating device 4 (optionally in wired or wireless communication with pressure generating device 4) or alternatively as a portion of pressure generating device 4 controlling operation(s) of pressure generating device 4.

Having thus described a general system in accordance with an example embodiment of the disclosed concept, an example of a method of using such a system will now be described. Referring to the flowchart of FIG. 3, initially movement and/or deformation of mask/patient interface 6 occurs that results in agitation of the triboelectric sensor(s) 10 positioned thereon, such as shown at 20, causing such sensor(s) 10 to generate a signature triboelectric signal, such as shown at 22. Such movement and/or deformation may occur when patient interface 6 is first placed on the head of patient P and/or during use of patient interface 6 when receiving treatment. As shown at 24, the signature triboelectric signal is acquired by controller 12 (e.g., via wired or wireless means). Controller 12 then compares the signature triboelectric signal to a database of known signals (e.g., stored in memory portion 16 locally or remotely) such as shown at 26 to find a corresponding signal in the database. As shown at 28, from such comparison at 26, controller 12 then determines information about the mask/patient interface 6 and then provides output based on such determination, such s shown at 30, and discussed in further detail below in conjunction with some detailed example embodiments. As discussed further below, such information about the mask/patient interface device may include, for example, without limitation, one or more of: the style of the patient interface device (e.g., nasal mask, oral/nasal mask, etc.), the size of the mask, condition of a portion of the mask, etc.

Having thus described an example system and method in accordance with the disclosed concept some detailed example embodiments will now be described.

### Embodiment 1: Triboelectric Contact Mode Signal Between Mask Elements

In this embodiment the triboelectric material combination is positioned between two of the mask elements, which move with respect to each other in a repeatable manner when the mask is used. The signal can be transferred to a processing unit on the mask or on the device, using wireless or wired communication. Some examples of this embodiment are given below, but the embodiment can be composed of any possible combination of the elements comprising the examples below.

### Example 1: Sliding Mode Signal Generation with Wired Connection to the Device

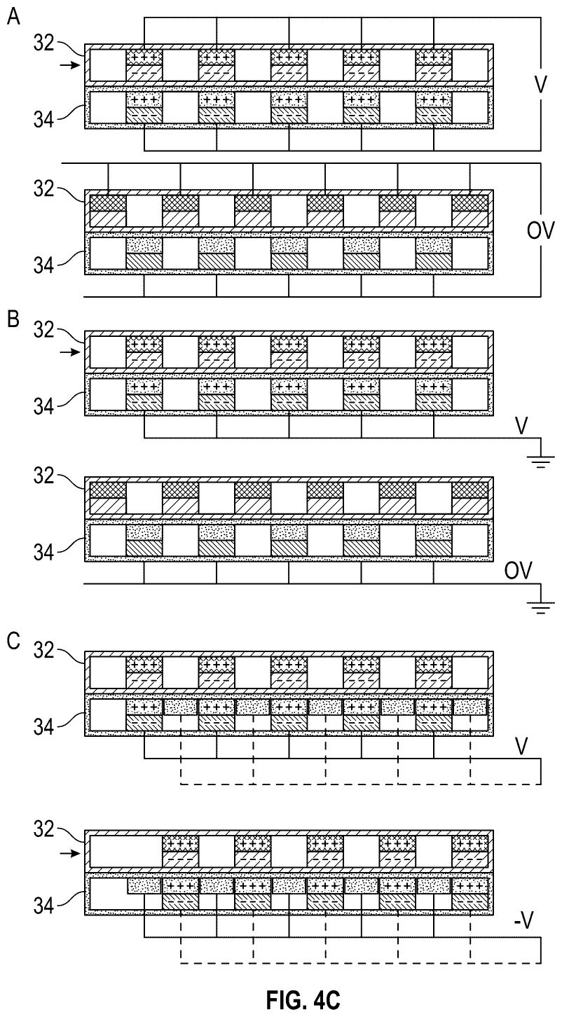

In this example, the triboelectric signal is generated by two materials in the patient interface. Sliding parts are configured with electrodes for signal acquisition. To enhance the effect, the electrodes may be configured in an alternating pattern, such as to generate a specific signal in response to the sliding motion. For instance, as shown in FIGS. 4A and 4B, two rings 32 and 34, located on mating surfaces in a hose swivel connection 36 may be equipped with alternating patterns of triboelectric materials consisting of a triboelectric series. The filler material between such patterns is a neutral material. When the swivel connection 36 turns, the opposite contact sides rotate with respect to each other and a triboelectric signal is generated.

For instance, in one configuration A as shown in FIG. 4C, one contact side is free to rotate, while the other part is fixed. Each rotation results in a signal generation depending on the precise combination of materials and electrode configuration. This signal has a good repeatability because of the controlled configuration of the two elements. For configurations B and C of FIG. 4C, the electrical connection for readout is only at the hose end, facilitating engineering aspects. This would be an ideal embodiment as it uses the movement of the hose relative to the patient interface for identification, because an easy way to pass a wire to the end of the hose using the helix already exists in the current products and this solution is free from any wires or other electronics in the patient interface.

For instance, a low number of elements will result in a relatively high voltage with a low frequency, whereas a large number of small elements will give a small signal that oscillates more rapidly. The absolute value of the amplitude and/or frequency characterizes the mask type and size. Optionally, additional electronic elements (RLC elements) can be added in the circuitry to manipulate the triboelectric signal and facilitate proper identification of the patient interface.

### Embodiment 2: Contact Separation Mode Signal with Wireless Connection to the Device

In this example, a different triboelectric mode is employed to generate a signal when the patient interface is put on by the patient and/or if a part of the patient interface is exchanged. For instance, the clip connection between headgear and a mask cushion may comprise a triboelectric sensor arrangement. Each time the clip is fastened to the cushion a signal may be generated by triboelectric charging via the contacting surfaces. The signal may be transferred via a wire to the controller where the magnitude of the signal governs the ID of the patient interface, as in the above example. Alternatively, the data is transferred via a wireless connection through a wireless transmission coil.

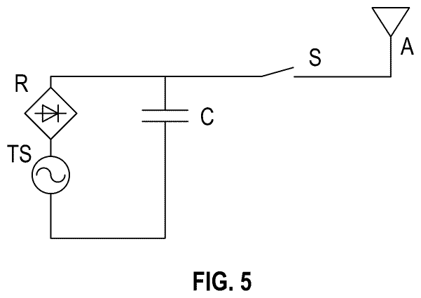

The triboelectric sensor can be coupled to the wireless coil antenna directly and may include simple electronic passive components that control the output signal. For instance by changing the value of a capacitor in parallel to the triboelectric sensor, the output frequency can be adapted to identify the patient interface. This method works best if the triboelectric sensor can generate enough power from a single movement. Alternatively, some additional simple electronics can be added to improve the quality of the transmitted signal. As an example, a switch is connected in series with the triboelectric system and a transmission coil. The triboelectric sensor generates a voltage on the buffer capacitor until switch voltage is reached. Then a controlled current will pass through the wireless antenna coil which sends a signal that can be picked up by a receiver in the PAP device or other suitable location. An example of such a circuit is illustrated in FIG. 5. Here, the self-powered triboelectric sensor TS provides a voltage which is (optionally) connected to a rectifier R and a buffer capacitor C. A switch S controls the voltage output of the signal that is passed through an antenna A. The buffer capacitor C may increase the power of the transmitted signal. The signal may be picked up by a coil on the PAP device and the patient interface may be identified from the characteristics of the discharge. The characteristics of the transmitted signal, such as frequency, amplitude, decay time or any combination of these may serve to identify the patient interface. In essence, all places where connections between mask elements are made can be a suitable location for such embodiment, such as, without limitation: mask clips, headgear straps, swivel connections, and cushion airpath connections.

In another example the triboelectric contact separation mode generates a signal when the triboelectric sensor part is put into contact with the human skin, for instance when the patient puts the patient interface on before use. The triboelectric sensor(s) are ideally located close to the patient facing surfaces, such as in the mask cushion or on the headgear (straps). This signal is registered by the controller and (re-)identifies the patient interface each time. The absolute signal (voltage) can be a determinant of the mask ID, which can be driven for instance by variation of the size of the triboelectric sensor and/or number of triboelectric sensors connected in series or other electronic configurations, potentially including passive elements. However, this method may be susceptible to variation arising from changes in the state and/or speed of contact, patient's skin condition, contamination on the triboelectric sensor surface and/or environmental conditions over time. This influences the output of the triboelectric sensor, although the extent of the effect can be limited by smart design of the triboelectric element itself.

Another, more accurate method makes use of a sensor containing two separate triboelectric elements with a specified difference. These elements may differ in size, material type or electrode configuration. The two elements are characterized such that the relative difference is known with respect to the absolute magnitude of the signal. The relative difference between the two signals is used for identification of the patient interface. Ideally the relative difference stays the same across the range of signals. Alternatively, if needed, a lookup table containing the relative difference compared to the absolute signal magnitude of one of the sensors is used to correct the relative signal and identify the correct patient interface. The dual element sensor configuration also neutralizes variations caused by contamination of the triboelectric surface or changes in triboelectric materials properties (degradation) over time, provided that both elements are in close proximity. As discussed above, the data transfer can be wired or wireless using the same technology described there. As an example, two copper electrodes were placed on the back of a silicone forehead element of a Philips Respironics Comfort Full Mask. The stack of copper material on silicone is a triboelectric sensor when contacting a third material (for instance human skin). Here the size difference of the electrodes governs the relative voltage output, regardless of the triboelectric operating mode. By changing the area ratio of the electrodes and thereby the specific output ratio, the type and size of the patient interface can be identified. Alternatively, the triboelectric characteristics can be different for each material, creating a different relative signal ratio. The noise, typically from electromagnetic fields from the sensor leads will be comparable for both sensors such that the sensor output ratio from unwanted noise signals is comparable for each electrode.

In addition to, or instead of identifying the type, size etc. of a patient interface, triboelectric sensor arrangements such as described herein can also/instead be used to determine degradation of a patient interface or selected portion(s) thereof. This is accomplished in a controller by comparing the incoming signal from the triboelectric sensor(s) to a database of stored signals that correspond to certain types and levels of degradation of the patient interface or selected portion(s) thereof. If the signal matches a certain stored signal the controller can identify what type of degradation is present and notify the user or other person(s) accordingly, e.g., via output device 18. The triboelectric element(s) may be designed in such a way that the output signal is dependent on a certain type of degradation. Optionally, the wear-out status may also updated in a therapy monitoring system and if a change is detected this may be flagged to caregivers (clinicians, DMEs). The following are some non-limiting examples of arrangements for monitoring degradation in accordance with the disclosed concept.

### Embodiment 3: Triboelectric Sensor for Mask Cushion Degradation

Triboelectricity is known to be highly dependent on the surface state of the material, where changes in surface state can cause the signal to deteriorate. The magnitude of the effect is dependent on the materials and surface conditions of the triboelectric surface. In this embodiment, a predefined surface (nano-)texture is applied, for instance to the mask cushion that is touching the skin (or any other location where two materials are in sliding or moving contact with each other), for example via (laser-) texturing the mold. This nano-texture greatly increases the output of the triboelectric sensor using known structures such as, for example, without limitation, surface nano-cubes or -pyramids. The surface texture is designed in such a way that it will wear out over time due to repeated contact with the triboelectric counter-surface and/or contamination (particles) in a controlled way and at a rate which is similar to the degradation of the whole mask element. The change in output as a result of the surface degradation can be compared to sensor outputs with known wear-out states stored in a database. The output can be applicable to the voltage polarity, voltage amplitude, decay time and/or a combination of any of these parameters. The wear out rates corresponding to the current state of the mask element can be communicated to the patient (e.g., via an output device), so the patient knows how many days are left until their patient interface or selected portion(s) thereof need(s) changing.

The signal can be controlled for instance by patterning the surface hierarchically, because patterns at different length scales (nm, μm) will wear out in different timescales. The different features, each corresponding to a known output voltage will wear out at different times, hence the output voltage will provide a means to estimate the wear-out of the surface (note wear-out mechanism could be due to material wear, surface deformation or filling of the gaps with contaminants and the patterns and materials need to be selected carefully to ensure which mechanism will dominate).

As an additional mechanism for determining mask wear-out, the fact that UV irradiation increases the triboelectric output in silicone rubber can be used. This effect can be used to determine the deterioration of the mask element as a result of UV exposure. A part of the sensor is surface textured as explained above whereas another part of the sensor is not textured (i.e., a separate electrode with separate voltage output). In this way the effect of UV degradation can be separated from the surface wear-out effect as described above. This facilitates wear-out monitoring based on different causes. For instance, for a mask that is heavily used, wear out will be detected based on a surface texture sensor whereas a mask that is left unused at the bedside but is left in a location that is exposed to sunlight during the day will be affected by the UV-A rays that largely pass through glass windows and affect the triboelectric properties of that part of the sensor. Alternatively, prolonged stress loading of the mask element may cause material degradation (cracking) in the silicone rubber which may cause a difference in voltage output due to changes in material structure and in surface and bulk properties which affect the triboelectricity generation of the sensor. This will generally have a negative effect on the triboelectric voltage generation, especially in combination with environmental contamination or water ingress.

### Embodiment 4: Triboelectric Sensor Voltage Output for Mask Contamination

Alternatively, the sensor output can be affected by surface contamination. The effect of contamination on the triboelectric sensitivity of the material is pronounced. This embodiment consists of a triboelectric sensor in contact with the skin, for instance in the mask cushion. The surface contamination such as skin oil, sebum, particles and other environmental aspects will affect the output of the triboelectric sensor. The sensor output can be compared to known signals from contaminated sensors to determine the amount of contamination. The output can be applicable to the voltage polarity, voltage amplitude, decay time and/or a combination of any of these parameters. The contamination level can be communicated to the patient to notify them of any necessary cleaning of the mask element. Ideally, the triboelectric material will recover its output after cleaning.

Additionally, the number of cleaning cycles may be stored in the device and/or the controller associated with the device, and an alert to replace the patient interface may be given to the patient when a predetermined number of cycles has been exceeded. Ideally, the triboelectric sensor output is almost, but not fully recovered after cleaning and will slowly deteriorate with the number of cleaning cycles. When the sensor fails to recover after a certain number of cleaning cycles, an alert to replace the mask may be given to the patient. Alternatively, the sensor consists of two elements, one (sealed) reference element which has a stable output over time and an element that is sensitive to contamination. The relative difference in output of the sensors can determine the state of contamination of the mask surface, which may allow a more accurate monitoring of the degradation by reducing the variance compared to using absolute output signals only. Efficient, demand driven mask cleaning can also be seen as a sustainability element as washing is a process that has a big CO2 footprint and personalized washing can reduce that footprint.

### Embodiment 5: Triboelectric Sensor Array with Different Sensitivities to Different Degradation Types

Ideally, the wear-out sensor element consists of a sensor array of triboelectric elements, each with a dependency on a different aspect of wear-out as explained in the previous embodiments. The following elements and aspects can be combined (in brackets the required configuration of the sensor): UV exposure (sealed from environment and without surface texture); Surface contamination and/or wear out (open and with surface texture); Stress cracking (sealed or open and with or without surface texture); Creep deformation (will alter the pitch of a surface texture, and the contact pressure); Reference element (sealed with UV blocking layer, no surface texture). Furthermore, potentially the triboelectric sensor can detect detachment of parts (e.g., foam) that was glued to a surface in a hollow structure, resp. a part which is moved by a changing airstream in the hollow lumen structure. By comparing the specific sensor readout to the reference element the different wear-out mechanisms due to each exposure type and level can be monitored.

Additionally, the charge output of the sensor during use may be stored locally in a battery and used to power a contamination or wear out indicator (e.g. flashing LED or beeper) that may be provided on the patient interface or other suitable location. Triboelectric sensors can generate enough energy to power flashing LEDs. This is advantageous as the patient interface does not require external power.

From the foregoing, it is thus to be appreciated that embodiments of the disclosed concept provide arrangements for detecting/determining information about a patient interface and/or portions thereof that improve upon and differ from known approaches.

While specific embodiments of the disclosed concept have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of disclosed concept which is to be given the full breadth of the claims appended and any and all equivalents thereof.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

What is claimed is:

1. A patient interface for use in providing a flow of a pressurized treatment gas to an airway of a patient, the patient interface comprising:
a body structured to receive the flow of the pressurized treatment gas;
a sealing member structured to engage with the face of the patient about the airway of the patient; and
at least one triboelectric sensor structured to provide a signal indicative of information about one or more of the body and/or the sealing member.

2. The patient interface of claim 1, wherein the at least one triboelectric sensor is structured to provide the signal indicative of information about one or more of the body and/or the sealing member when a portion of the patient interface moves with respect to another portion of the patient interface or with respect to the face of the patient.

3. The patient interface of claim 1, wherein the signal is indicative of a type of patient interface or a portion thereof.

4. The patient interface of claim 1, wherein the signal is indicative of a size of the patient interface or a portion thereof.

5. The patient interface of claim 1, wherein the signal is indicative of a condition of a portion of the patient interface.

6. The patient interface of claim 1, further comprising an antenna in communication with the at least one triboelectric sensor.

7. The patient interface of claim 1, wherein the at least one triboelectric sensor comprises a plurality of triboelectric sensors.

8. The patient interface of claim 7, wherein at least two triboelectric sensors of the plurality of triboelectric sensors are structured to provide a signal indicative of the same information.

9. The patient interface of claim 7, wherein at least two triboelectric sensors of the plurality of triboelectric sensors are structured to provide a signal indicative of different information.

10. A system for providing a flow of a pressurized treatment gas to an airway of a patient, the system comprising:
a pressure generating device structured to produce a flow of a pressurized treatment gas;
a patient interface coupled to, and structured to receive the flow of pressurized treatment gas from, the pressure generating device via a conduit, the patient interface comprising:
a body structured to receive the flow of the pressurized treatment gas from the conduit;
a sealing member structured to engage with the face of the patient about the airway of the patient; and
at least one triboelectric sensor structured to provide a signal indicative of information about one or more of the body and/or the sealing member; and
a controller in communication with the at least one triboelectric sensor, the controller structured to receive the signal indicative of information about one or more of the body and/or the sealing member and determine therefrom the information about the one or more of the body and/or the sealing member.

11. The system of claim 10, wherein the at least one triboelectric sensor is structured to provide the signal indicative of information about one or more of the body and/or the sealing member when a portion of the patient interface moves with respect to another portion of the patient interface or with respect to the face of the patient.

12. The system of claim 10, wherein the signal is indicative of:
a type of patient interface or a portion thereof,
a size of the patient interface or a portion thereof, or
a condition of a portion of the patient interface.

13. The system of claim 10, further comprising an antenna in communication with the at least one triboelectric sensor.

14. The system of claim 10, wherein the at least one triboelectric sensor comprises a plurality of triboelectric sensors.

15. A method of determining information about a patient interface used in providing a flow of pressurized treatment gas to the airway of a patient, the method comprising:
receiving a signal from a triboelectric sensor provided on the patient interface;
comparing the signal to a database of known signals;
determining information about the patient interface from the comparison; and
providing an output based on the information.
